(19) United States
(12) Patent Application Publication
(10) Pub. No.: US 2024/0279374 A1
(43) Pub. Date: Aug. 22, 2024

(54) BIODEGRADABLE POLYMERS THAT ELUTE AN ACTIVE SUBSTANCE

(71) Applicant: 4D MEDICINE LIMITED, Birmingham, West Midlands (GB)

(72) Inventors: Andrew Peter DOVE, Birmingham, West Midlands (GB); Andrew Christopher WEEMS, Birmingham, West Midlands (GB)

(21) Appl. No.: 18/566,469

(22) PCT Filed: Apr. 1, 2022
(86) PCT No.: PCT/GB2022/050828

§ 371 Date: Dec. 1, 2023

(30) Foreign Application Priority Data
Jun. 4, 2021 (GB) 2107999.1

(51) Int. Cl. C08F 218/04; A61K 47/58; A61L 27/16; A61L 27/54

(52) U.S. Cl. CPC C08F 218/04; A61K 47/58; A61L 27/16; A61L 27/54; A61L 2300/41

(57) ABSTRACT

A photocurable resin composition, the photocurable resin composition comprises a photoinitiator, optionally an oligomer comprising at least one alkene moiety (which may be a 1,2-disubstitued moiety, or an acrylate moiety), and a drug-derived monomer (1, 2, 3, 4, 5) having at least one photo-crosslinkable moiety which may be an alkene moiety).

This invention relates generally to biocompatible polymers that elute an active substance, for example a drug molecule, upon degradation. More specifically, although not exclusively, this invention relates to a biocompatible polymer that elutes a NSAID (non-steroidal anti-inflammatory drug) upon degradation. The invention further relates to a resin composition for use in making the biocompatible polymer, and a method of making the same.

Biodegradable polymers are polymers that breakdown over a finite period of time after being placed in the body. It is preferable that the polymer itself, as well as the resultant break-down products are non-toxic. It is known to incorporate biodegradable polymers into drug delivery systems. Drug delivery systems offer advantages over conventional delivery routes including the ability to maintain a plasma concentration of the drug molecule above a therapeutic threshold. In contrast, an orally administered drug molecule may release a large amount of the active ingredient over a short period of time, followed by a rapid decline in the plasma concentration to below the therapeutic threshold. Oral medicines can be provided with enteric coatings to control in vivo dissolution rates but these can affect bioavailability.

Drug delivery systems comprising biodegradable polymers will often encapsulate or otherwise contain the drug molecule in a carrier matrix, for release as the carrier matrix either degrades by excretion and/or metabolism without substantial interaction with the host. In many cases, the release rate of the drug molecule is dependent upon the hydrophobicity of the polymer matrix, as well as the diffusion rates in the specific environment in which the drug delivery system is utilized, e.g. the blood or fat tissue.

Uhrich et al. has developed a series of biodegradable polymers for potential application in drug delivery systems. The biodegradable polymers developed in this work incorporate salicylic acid into a biodegradable polymer backbone (for example, see Uhrich et al. Biomacromolecules, 2005, 6, 359-367; Macromolecules, 2000, 33, 6217-6221; and US2012/0058155). Salicylate is the active metabolite of Aspirin (RTM), a well-known and widely used non-steroidal anti-inflammatory drug (NSAID). The Uhrich polymers undergo non-enzymatic hydrolytic degradation to release salicylic acid in a controlled manner. Advantageously, this overcomes the therapeutic threshold limit imposed by the body when too high a dose is introduced because the majority of salicylic acid is rapidly excreted and never reaches the desired location.

The poly(anhydride esters) developed by Uhrich et al. were synthesised by melt condensation polymerisation, for example, requiring the monomers to be heated to 180° C. These processing conditions represent the main limitation for use of the proposed system in the fabrication of medical devices and for biotechnology applications. It is difficult to process this type of polymer into a bespoke shape or geometry. This provides a challenge for wide-scale adoption because conventional manufacturing is desirous for cost reasons to produce medical-grade quality parts. Further, the biopolymers according to the Uhrich et al system are highly crystalline, glassy polymers, which are prone to fracturing which raises handling, deployment, and use issues

It is therefore a first non-exclusive object of the invention to provide a biodegradable polymer that elutes a drug, for example one or more of an NSAID, such as salicylic acid, a chemotherapeutic agent such as doxorubicin, an antibiotic, an anti-inflammatory agent or an analgesic, for use medical applications that has improved processability for manufacturing parts of specific and bespoke geometries.

A first aspect of the invention provides a photocurable resin composition, the photocurable resin composition comprising a photoinitiator, optionally an oligomer comprising at least one alkene moiety (e.g. a 1,2-disubstitued moiety, or an acrylate moiety), and a drug-derived monomer having at least one photo-crosslinkable moiety (for example an alkene moiety).

A further aspect of the invention provides a photocurable resin composition, the photocurable resin composition comprising a photoinitiator, optionally an oligomer comprising at least one alkene moiety (e.g. a 1,2-disubstitued moiety, or an acrylate moiety), and a drug-derived monomer having at least one photo-crosslinkable moiety (for example an alkene moiety), wherein the sum of alkene moieties on the optional oligomer and photo-crosslinkable moiety on the drug-derived monomer is two or greater, preferably greater than two.

In this specification the term acrylate is intended to incorporate methacrylates, ethacrylates and so on unless the context specifies otherwise.

In embodiments the drug-derived monomer may comprise two groups R¹ or R² and one or both of R¹ or R² may comprise an alkene moiety (e.g. a 1,2-disubstituted alkene, and/or an acrylate moiety).

In embodiments, the drug-derived monomer may be a monomer derived from one or more of the following NSAID classes: salicylates, propionic acid derivatives, acetic acid derivatives, enolic acid (oxicam) derivatives, anthranilic acid derivatives (fenamates), selective COX-2 inhibitors, sulfonanilides, and others.

The NSAID class “salicylates” includes the following NSAIDS: Aspirin (acetylsalicylic acid); Diflunisal (Dolobid); Salicylic acid and its salts; Salsalate (Disalcid).

The NSAID class “propionic acid derivatives” includes the following NSAIDS: Ibuprofen; Dexibuprofen; Naproxen; Fenoprofen; Ketoprofen; Dexketoprofen; Flurbiprofen; Oxaprozin; Loxoprofen.

The NSAID class “acetic acid derivatives” includes the following NSAIDS: Indomethacin; Tolmetin; Sulindac; Etodolac; Ketorolac; Diclofenac; Aceclofenac; Nabumetone.

The NSAID class “Enolic acid (oxicam) derivatives” includes the following NSAIDS: Piroxicam; Meloxicam; Tenoxicam; Droxicam; Lornoxicam; Isoxicam; Phenylbutazone (Bute).

The NSAID class “Anthranilic acid derivatives (fenamates)” are derived from fenamic acid, which is a derivative of anthranilic acid, which in turn is a nitrogen isostere of salicylic acid, which is the active metabolite of aspirin. This class includes the following NSAIDS: Mefenamic acid; Meclofenamic acid; Flufenamic acid; Tolfenamic acid.

The NSAID class “Selective COX-2 inhibitors (coxibs)” includes the following NSAIDs: Celecoxib; Rofecoxib; Valdecoxib; Parecoxib; Lumiracoxib; Etoricoxib; Firocoxib.

The NSAID class “Sulfonanilides” includes the NSAID Nimesulide.

The NSAID class defined as “others” includes the following NSAIDS: Clonixin; Licofelone; H-harpagide.

The drug may comprise a chemotherapeutic agent such as doxorubicin, an antibiotic, an anti-inflammatory agent or an analgesic.

Accordingly, a further or more specific aspect of the invention provides a photocurable resin composition, the photocurable resin composition comprising a photoinitiator, optionally an oligomer comprising at least one alkene moiety (e.g. a 1,2-disubstitued moiety, or an acrylate moiety), and an NSAID-derived monomer, the NSAID-derived monomer having the following general formula:

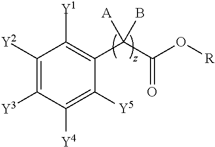

wherein Y¹, Y², Y³, Y⁴ and Y⁵ independently represent hydrogen or a halogen (e.g. fluorine, chlorine, bromine, or iodine), or may comprise carbon (e.g. an aliphatic group or chain, or an aromatic group or chain, for example a straight branched or cyclic alkane, or an aryl group), oxygen (e.g. a hydroxyl moiety, or a substituted oxygen moiety such as an ether, ester, ketone, aldehyde, amide and may be an acetyl moiety), sulphur (e.g. an SH thiol moiety, or a substituted thiol moiety), nitrogen (e.g. an NH₂ moiety, or a di- or tri-substituted nitrogen moiety) or Rₓ, C—Rₓ, O—Rₓ, C(O)—Rₓ where Rₓ represents a straight, branched or cyclic alkane (e.g. C1 to C6 alkane), which may comprise one or more hetero atoms or Rₓ may represent an aryl group, e.g. a phenyl group or substituted phenyl group;
A and B independently represent or comprise a hydrogen atom, a halogen atom, or an alkyl group (for example a C1 to C3 alkane, e.g. a methyl group);
z represents an integer of 0, 1, 2, 3, 4, or 5 and
R comprises a photo-crosslinkable moiety, for example an alkene moiety (e.g. a 1,2-disubstituted alkene, and/or an acrylate moiety).

Preferably, z is an integer of 0 or 1.

One or more of Y¹, Y², Y³, Y⁴ and Y⁵ may comprise a photocrosslinkable group, for example an alkene moiety (e.g. a 1,2-disubstituted alkene, and/or an acrylate moiety), in embodiments Y⁵ comprises a photocrosslinkable group, for example an alkene moiety (e.g. a 1,2-disubstituted alkene, and/or an acrylate moiety). In embodiments, Y¹, Y², Y³ and Y⁴ do not comprise a photocrosslinkable group.

In an embodiment the sum of alkene moieties on the optional oligomer and photo-crosslinkable moieties on the drug-derived monomer is two or greater, preferably greater than two.

For example, the NSAID-derived monomer may be a derivative of one of Aspirin (acetylsalicylic acid); Diflunisal (Dolobid); Salicylic acid and its salts; Salsalate (Disalcid); or Ibuprofen; Dexibuprofen; Naproxen; Fenoprofen; Ketoprofen; Dexketoprofen; Flurbiprofen; Loxoprofen.

If the NSAID-derived monomer is proprionic acid derivative (for example ibuprofen or one of Dexibuprofen; Naproxen; Fenoprofen; Ketoprofen; Dexketoprofen; Flurbiprofen; Loxoprofen) Y³ may comprise one of Rₓ, C—Rₓ, O—Rₓ, C(O)—Rₓ where Rₓ represents a straight, branched or cyclic alkane (e.g. C1 to C6 alkane), which may comprise one or more hetero atoms or Rₓ may represent an aryl group, and/or each of Y¹, Y², Y⁴ may independently be hydrogen or a halide, z may be 1 and A and B may represent hydrogen and a methyl group.

In an embodiment Y⁵ may comprise a photocrosslinkable group, for example an alkene moiety (e.g. a 1,2-disubstituted alkene, and/or an acrylate moiety. Y⁵ may be represented by the group O—R² wherein R² may comprise a photocrosslinkable group, for example an alkene moiety (e.g. a 1,2-disubstituted alkene, and/or an acrylate moiety.

Accordingly, a further or more specific aspect of the invention provides a photocurable resin composition, the photocurable resin composition comprising a photoinitiator, optionally an oligomer comprising at least one alkene moiety (e.g. a 1,2-disubstitued moiety, or an acrylate moiety), and a salicylic acid derived monomer, the salicylic acid derived monomer having the following general formula:

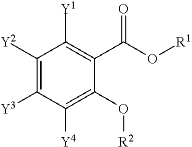

wherein Y¹, Y², Y³, and Y⁴ independently represent or comprise hydrogen, carbon (e.g. an aliphatic group or chain, or an aromatic group or chain), a halogen (e.g. fluorine, chlorine, bromine, or iodine), oxygen (e.g. a hydroxyl moiety, or a substituted oxygen moiety such as an acetyl group or ether), sulphur (e.g. an SH thiol moiety, or a substituted thiol moiety), or nitrogen (e.g. an NH₂ moiety, or a di or tri-substituted nitrogen moiety);
and one or both of R¹ or R² comprise photocrosslinkable moiety, for example an alkene moiety (e.g. a 1,2-disubstituted alkene moiety, and/or an acrylate moiety).

In an embodiment the sum of alkene moieties on the optional oligomer and photo-crosslinkable moieties on the drug-derived monomer is two or greater, preferably greater than two.

In embodiments, one or more (e.g. two) of Y¹, Y², Y³, and Y⁴ may combine together to form a condensed ring (e.g. a condensed aromatic ring).

Advantageously, the crosslinked polymers fabricated from the resin compositions according to the invention are designed to degrade in vivo over a finite period of time to elute a drug, e.g. a salicylic acid derivative, which is usable by the human or animal body as a drug or prodrug, e.g. a drug or prodrug for salicylic acid.

In addition, the thermomechanical properties, the degradation rates, and the shape memory response of the resulting crosslinked polymers may be tuned by adjusting the quantities of the components of the photocurable resin composition.

More advantageously, the photocurable resin composition according to the invention are photocurable. The alkene functionality on the oligomer as well as the functionality of the monomer on the R¹ and/or R² groups are able to form covalent bonds with species in the resin formulation such that a crosslinked polymer is produced. The ability of the resin composition to photocure means that elevated temperatures are not required to polymerise the resin composition, for example, via melt condensation. This allows the resin composition to be processed and implemented under milder conditions. In addition, the resin compositions according to the invention have low viscosities, which allow them to readily flow before being photo-crosslinked into soft, flexible solid parts.

Moreover, the photocurable resin composition according to the invention may be used to produce 3D objects, for example, using additive manufacture with a light source, e.g. 3D printing. The 3D object may comprise a solid object, e.g. a porous object, a film, and/or a hydrogel polymer system. Additive manufacture using photocuring may be used to manufacture objects comprising specific and complex geometries, for example, for use in biomedical applications.

In embodiments, R¹ and/or R² may comprise between 1 to 20 carbon atoms, e.g. between 1 to 15 carbon atoms, or 1 to 10 carbon atoms, for example, the R¹ and/or R² may comprise 1, 2, 3, 4, 5, 6, 7, 8, 9, or 10 carbon atoms. In embodiments, R¹ and/or R² may comprise an alkene moiety, for example, a 1,2-disubstituted alkene, an acrylate moiety, a trisubstituted alkene moiety, and/or a tetra-substituted alkene moiety. In embodiments, the R¹ and/or R² may comprise a carbonyl group, e.g. a ketone, an ester, a carbonate, an amide, and/or a thioester. In embodiments, the R¹ and/or R² may comprise halogen atoms, e.g. fluorine, chlorine, bromine, or iodine. In embodiments, the R¹ and/or R² may comprise hetero-atoms, e.g. nitrogen, oxygen, sulphur, phosphorus. Additionally or alternatively, R¹ and/or R² may comprise a halogen atom, e.g. a fluorine, chlorine, bromine, and/or iodine atom.

In embodiments R¹ and/or R² may be branched or may comprise a straight chain, e.g. of carbon atoms.

In embodiments R¹ and/or R² may comprise an aryl moiety, the aryl moiety may comprise a substituted aryl moiety, e.g. a substituted benzyl moiety. In embodiments, the aryl moiety may comprise a branched alkyl portion, or a straight alkyl chain portion, e.g. of carbon atoms.

In embodiments, the salicylic acid derived monomer has the following general formula:

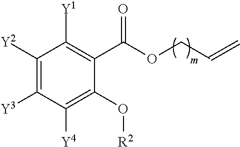

wherein m is a number between 1 and 5;
Y¹, Y², Y³, Y⁴, and R² are defined as previously specified.

In embodiments, m is a number between 1 and 4, or 1 and 3, or 1 and 2, e.g. m=1, 2, 3, 4, or 5. In embodiments, m is 1.

In embodiments, R² represents hydrogen. For example, the salicylic acid derived monomer may have the following general formula:

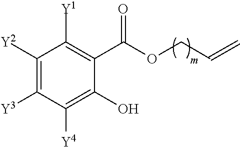

wherein Y¹, Y², Y³, Y⁴, and m are defined as previously specified.

In embodiments, the salicylic acid derived monomer has the following general formula:

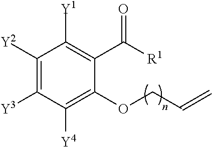

wherein n is a number between 1 and 5;
Y¹, Y², Y³, Y⁴, and R¹ are defined as previously specified.

In embodiments, n is a number between 1 and 4, or 1 and 3, or 1 and 2, e.g. n=1, 2, 3, 4, or 5. In embodiments, n is 1.

For example, the salicylic acid derived monomer may have the following general formula:

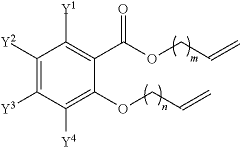

wherein m, n, Y¹, Y², Y³, and Y⁴ are defined as previously specified.

In embodiments, the salicylic acid derived monomer has the following general formula:

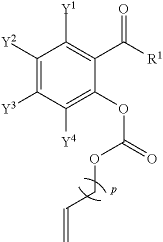

wherein p is a number between 1 and 5;
Y¹, Y², Y³, Y⁴, and R¹ are defined as previously specified.

In embodiments, p is a number between 1 and 4, or 1 and 3, or 1 and 2, e.g. p=1, 2, 3, 4, or 5. In embodiments, p is 1.

For example, the salicylic acid derived monomer may have the following general formula:

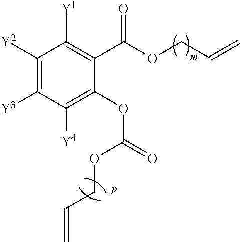

wherein m, p, Y¹, Y², Y³, and Y⁴ are defined as previously specified.

In embodiments, Y¹, Y³, and Y⁴ each independently represent hydrogen, and Y² represents one of hydrogen, bromine, or iodine. The salicylic acid derived monomer may comprise the following general formula:

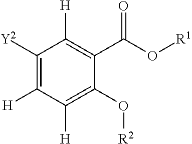

wherein R¹ and R² are defined as previously specified in any embodiment; and
Y² represents one of hydrogen, bromine, or iodine.

In embodiments, the salicylic acid derived monomer may be selected from one or more of the following compounds (i) to (v):

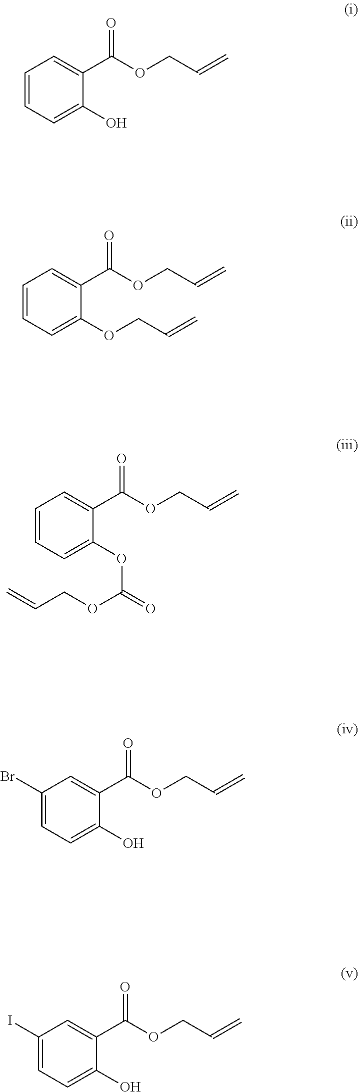

Monomers (i), (iv), and (v) are known to form pendant salicylic acid moieties within the crosslinked polymer. It has been found that these monomers may be used to achieve rapid release and mass-loss of the part, as well as having a more rapid thermomechanical integrity reduction.

In contrast, Monomers (ii) and (iii) may form in-chain salicylic acid moieties within the crosslinked polymer, and be used to achieve slower, longer release rates without altering the final dose of salicylic acid that may be obtained, e.g. by using a greater than 50 wt. % loading by mass of the final part.

In embodiments, the photocurable resin composition may comprise more than one distinct a salicylic acid derived monomer, e.g. two or more different a salicylic acid derived monomers.

In embodiments wherein the oligomer is present, the one or more salicylic acid derived monomer(s) may be present in up to 80 wt. % of the total resin composition. For example, the one or more salicylic acid derived monomer(s) may be present in between greater than 0 and less than 80 wt. %, for example, between 5 and 70 wt. %, or between 10 to 60 wt. %, or between 20 to 50 wt. %, or between 30 to 40 wt. % of the total resin composition. For example, the one or more salicylic acid derived monomer(s) may comprise between any one of 5, 10, 15, 20, 25, 30, 35, 40, 45, 50, 55, 60, 65, 70, 75, 80 wt. % to 75, 70, 65, 60, 55, 50, 45, 40, 35, 30, 25, 20, 15, or 10 wt. % of the total resin composition. In embodiments, the one or more salicylic acid derived monomer(s) may comprise between 40 to 70 wt. % of the total resin composition. In embodiments, the one or more salicylic acid derived monomer(s) are present in up to 50 wt. % of the total resin composition.

In embodiments, the oligomer may not be present. In embodiments, the oligomer is present in up to 50 wt. % of the total resin composition. For example, the oligomer may be present in between any one of 0, 5, 10, 15, 20, 25, 30, 35, 40, or 45 wt. % to any one of 50, 45, 40, 35, 30, 25, 20, 15, 10 or 5 wt. % of the total resin composition. In embodiments comprising oligomers with pendant salicylic acid derived groups (e.g. Monomer i, Monomer iv, or Monomer v), the oligomer may be present in up to 20 wt. % the total resin composition. In embodiments comprising oligomers with in-chain salicylic acid derived groups (e.g. Monomer ii or Monomer iii), the oligomer may not be present, or alternatively may be present in up to 20 wt. % of the total resin composition.

In embodiments, the oligomer comprises one or more polycarbonate linkages. In embodiments, the oligomer consists of polycarbonate linkages along a carbon backbone chain. In embodiments, the photocurable resin composition comprises more than one type of oligomer, e.g. a further oligomer comprising one or more of a polyester linkage, or a polyurethane linkage.

In embodiments, the oligomer is fabricated from a monomer with the following structure:

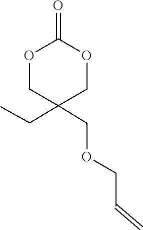

In embodiments, the oligomer is fabricated from a monomer with the following structure:

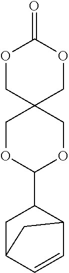 [SMILES: O=C1OCC2(CO1)COC(C1CC3C=CC1C3)OC2]

In embodiments, the oligomer comprises between 3 and 100 repeating monomers.

Whilst the above has been discussed in reference to salicylic acid monomers, the same may be true of other NSAID monomers.

For example, the monomer may be one or more of:

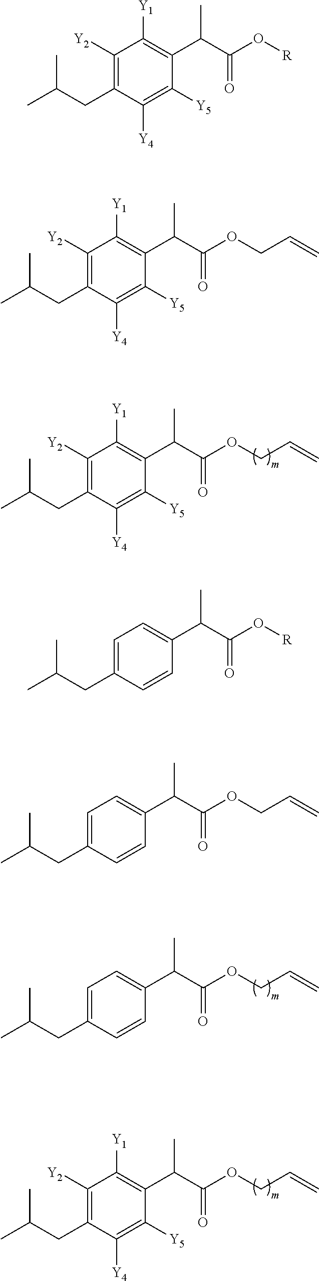

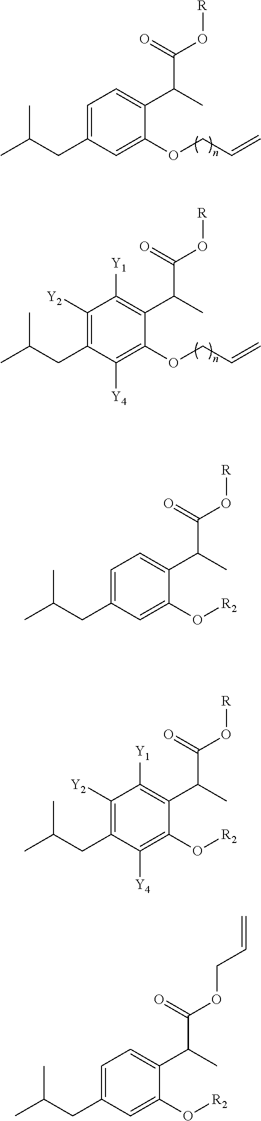

where Y¹, Y², Y⁴, Y⁵, R and R² are as previously described, m may be an integer from 1 to 5, n may be an integer of from 1 to 5 and the (Y³) isobutyl moiety may be replaced by C—Rₓ, O—Rₓ, C(O)—Rₓ where Rₓ represents a straight, branched or cyclic alkane (e.g. C1 to C6 alkane), which may comprise one or more hetero atoms or Rₓ may represent an aryl group, e.g. a phenyl group or substituted phenyl group.

For example where R and R² both comprise photocrosslinkable moieties the monomer may form in-chain moieties whereas those monomers with single phtocrosslinkable groups may be able to form pendant moieties with eh polymer backbone.

A further aspect of the invention may provide a photocurable resin composition, the photocurable resin composition comprising a photoinitiator, an oligomer comprising at least one alkene moiety (e.g. a 1,2-disubstitued alkene moiety, or an acrylate moiety), and an NSAID-derived monomer, the NSAID-derived monomer may be selected from one of more of the acetic acid derived NSAIDs, the enolic acid derived NSAIDs, the anthrilic acid derived NSAIDs, for example one or more of:

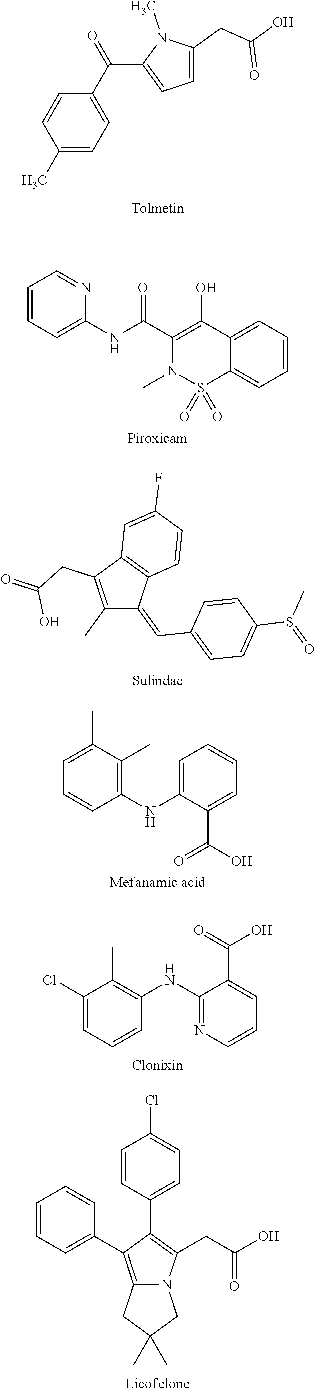 [SMILES: Cc1ccc(C(=O)c2ccc(CC(=O)O)n2C)cc1 ; CN1C(C(=O)Nc2ccccn2)=C(O)c2ccccc2S1(=O)=O ; CC1=C(CC(=O)O)c2cc(F)ccc2/C1=C\c1ccc(S(C)=O)cc1 ; Cc1cccc(Nc2ccccc2C(=O)O)c1C ; Cc1c(Cl)cccc1Nc1ncccc1C(=O)O ; CC1(C)Cc2c(-c3ccccc3)c(-c3ccc(Cl)cc3)c(CC(=O)O)n2C1]

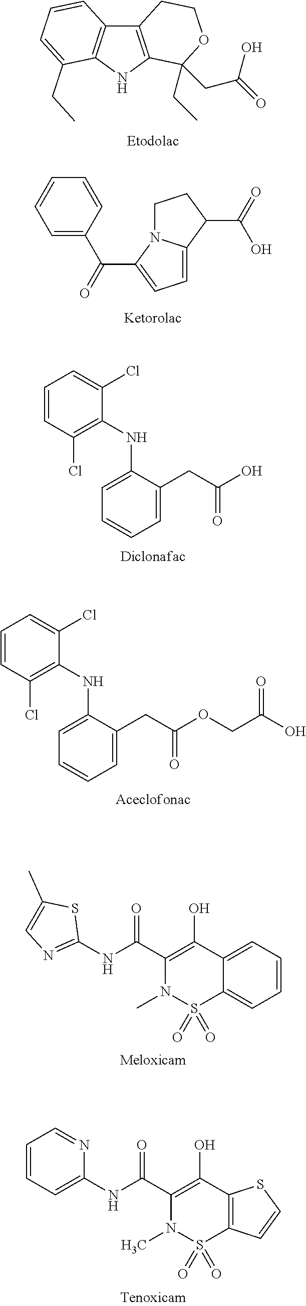 [SMILES: CCc1cccc2c3c([nH]c12)C(CC)(CC(=O)O)OCC3 ; O=C(c1ccccc1)c1ccc2n1CCC2C(=O)O ; O=C(O)Cc1ccccc1Nc1c(Cl)cccc1Cl ; O=C(O)COC(=O)Cc1ccccc1Nc1c(Cl)cccc1Cl ; Cc1cnc(NC(=O)C2=C(O)c3ccccc3S(=O)(=O)N2C)s1 ; CN1C(C(=O)Nc2ccccn2)=C(O)c2sccc2S1(=O)=O]

wherein in each case a or the hydroxyl group has been functionalised by a photocrosslinkable moiety such as an alkene or acrylate moiety.

In embodiments, the photoinitiator may be a radical initiator, that is a photoinitiator that generates radicals upon exposure to light. In embodiments, the photoinitiator may comprise Irgacure 819 (RTM) manufactured by Ciba Speciality Chemicals (acquired by BASF, Basel, Switzerland), that is bis(2,4,6-trimethylbenzoyl)-phenylphosphineoxide having the following chemical formula:

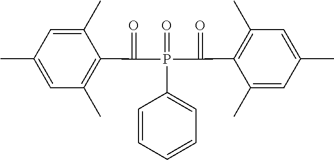

In embodiments, the photoinitiator may comprise Irgacure 369 (RTM) manufactured by Ciba Speciality Chemicals (acquired by BASF, Basel, Switzerland), that is 2-benzyl-2-dimethylamino-1-(4-morpholinophenyl)-butanone-1 having the following chemical formula:

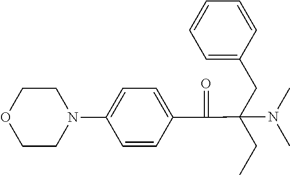 [SMILES: CCC(Cc1ccccc1)(C(=O)c1ccc(N2CCOCC2)cc1)N(C)C]

In embodiments, the photoinitiator is present in up to 1.0 wt. %, or 0.75 wt. %, or 0.5 wt. %, for example, up to 0.4 wt. %, or up to 0.3 wt. %, or up to 0.2 wt. %, or up to 0.1 wt. % of the total resin composition. Preferably, the photoinitiator is present in between 0.2 to 0.4 wt. %.

In embodiments, the photoinitiator may comprise a free radical initiator. In embodiments, the photocurable resin composition may comprise a multifunctional acrylate to set crosslink density, for example, in acrylate free radical polymerization.

In embodiments, the photocurable resin composition further comprises a photoinhibitor. In embodiments, the photoinhibitor may be paprika extract. The photoinhibitor may be present in up to 1.0 wt. %, or 0.75 wt. %, or 0.5 wt. %, for example, up to 0.4 wt. %, or up to 0.3 wt. %, or up to 0.2 wt. %, or up to 0.1 wt. % of the total resin composition. Preferably, the photoinitiator is present in between 0.1 to 0.4 wt. %.

In embodiments, the photocurable resin composition comprises a crosslinker. In embodiments, the crosslinker may comprise a compound comprising two or more thiol moieties, e.g. a 4-armed thiol crosslinker such as pentaerythritol tetrakis(3-mercaptopropionate).

Advantageously, in embodiments of the invention comprising a crosslinker comprising thiol moieties, the alkene moieties of the oligomer and/or salicylic acid based monomer are able to undergo thiol-ene click chemistry when exposed to UV light, to produce the crosslinked polymer. More advantageously, this may be readily implemented on a 3D printer to produce intricate and bespoke objects. The geometry of the object may also be designed to enable timed release of the salicylic acid as the object degrades in vivo.

In embodiments, the photocurable resin composition may comprise, or further comprise the following photoinitiator:

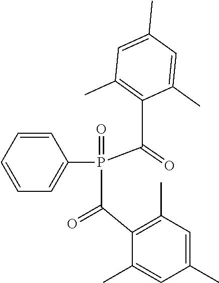 [SMILES: Cc1cc(C)c(C(=O)P(=O)(C(=O)c2c(C)cc(C)cc2C)c2ccccc2)c(C)c1]

In embodiments, the photocurable resin composition may further comprise a diluent, e.g. propylene carbonate.

A further aspect of the invention provides a crosslinked polymer fabricated from any of the photocurable resin compositions according to invention. In embodiments, the crosslinked polymer may be used as a scaffold for a biomedical application.

Advantageously, the crosslinked polymer may further exhibit shape memory properties. This enables an article, e.g. a scaffold, fabricated from the crosslinked polymer to be repeatedly deformed without damage.

A further aspect of the invention provides a method of crosslinking a resin composition comprising drug-derived monomers to form a polymer, the method comprising providing a comprising a photoinitiator, optionally an oligomer comprising at least one alkene moiety (e.g. a 1,2-disubstitued moiety, or an acrylate moiety), and a drug-derived monomer having at least one photocross-linkable moiety (for example an alkene moiety) and irradiating the composition.

In an embodiment the sum of alkene moieties on the optional oligomer and photo-crosslinkable moieties on the drug-derived monomer is two or greater, preferably greater than two.

A yet further aspect of the invention provides a method of crosslinking a resin composition comprising salicylic acid derived monomers, to form a polymer, the method comprising:
i. providing a photocurable resin composition, the photocurable resin composition comprising optionally an oligomer comprising an alkene moiety (e.g. a 1,2-disubstitued moiety, or an acrylate moiety), a photoinitiator, and an NSAID derived monomer, for example a salicylic acid derived monomer having the following general formula:

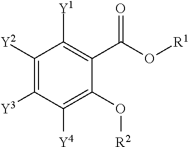

wherein Y¹, Y², Y³, and Y⁴ independently represent hydrogen, a halogen (e.g. fluorine, chlorine, bromine, or iodine), oxygen (e.g. a hydroxyl moiety, or an ether), or nitrogen (e.g. an NH₂ moiety, or a di- or tri-substituted nitrogen moiety);
R¹ and R² are independently an alkyl or aryl moiety;
and one or both of R¹ or R² comprise an alkene moiety (e.g. a 1,2-disubstituted alkene, and/or an acrylate moiety).
ii. irradiating the resin composition with light.

In embodiments, step ii. of the method comprises irradiating the resin composition with UV light.

In embodiments, the method may be performed by additive manufacturing, e.g. on a 3D printer.

The method may further comprise addition of an active to the photocurable resin composition for encapsulation in the crosslinked polymer. The active may comprise a further drug molecule, for example, another NSAID such as ibuprofen. The active may comprise free salicylic acid, i.e. not bonded to the crosslinked polymer matrix.

A yet further aspect of the invention provides a crosslinked polymer fabricated using the method according to the invention.

In embodiments, the crosslinked polymer may further comprise or contain a further active, e.g. a drug molecule, for example, a further anti-inflammatory drug molecule such as Ibuprofen or free salicylic acid. Advantageously, the crosslinked polymer may contain further drug molecules for controlled release as the crosslinked polymer degrades. In embodiments, the further drug molecule is free within the crosslinked polymer matrix, that is, the further drug is not covalently bonded to the crosslinked polymer matrix. In embodiments, the crosslinked polymer may comprise distinct layers, e.g. each comprising a different type or concentration of free active within the matrix.

Advantageously, the invention may be implemented such that a chemical crosslink is established using a specific wavelength of light to initiate a specific reaction (e.g. thiol-ene click chemistry crosslinking) to produce a crosslinked polymer comprising a salicylic acid derived monomer bonded within the crosslinked matrix, and a further active (e.g. free salicylic acid or a different active such as doxorubicin) which is contained within (but not covalently bonded to) the crosslinked polymer matrix. Upon degradation, the salicylic acid derived monomer and the further active are released at the same rate of the surface erosion takes place, which overcomes both the sustained release limitations of loaded systems as well as loading limitations where the salicylic acid, may act as a plasticizer.

More advantageously, the invention may be implemented to form a crosslinked polymer article comprising multiple layers of polymer with different types and/or concentration of active in each layer. In this way. salicylic acid is constantly released along with selected drug species interspaced throughout the printed part. This allows for development of designer polypills containing multiple drugs either spatially controlled, regionally selected, or homogenously blended. This type of approach could be useful for dual-drug therapies, or to ensure that a specific drug-regimen is followed for treatment.

Furthermore, the method of the invention may be implemented using standard stereolithographic printers. Moreover, post polymerization procedures may be carried out on the resulting crosslinked polymer article using a second curing chamber. For example, two photoinitiators may be added to the photocurable composition, the photoinitiators having two different absorption bands. Selective patterning may be achieved by varying the wavelengths of light to form the 3D printed part. This method may be used to form tissue scaffolds or advanced medical devices.

Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible. For the avoidance of doubt, the terms “may”, “and/or”, “e.g.”, “for example” and any similar term as used herein should be interpreted as non-limiting such that any feature so-described need not be present. Indeed, any combination of optional features is expressly envisaged without departing from the scope of the invention, whether or not these are expressly claimed. The applicant reserves the right to change any originally filed claim or file any new claim accordingly, including the right to amend any originally filed claim to depend from and/or incorporate any feature of any other claim although not originally claimed in that manner.

To further exemplify the invention, reference is made to the following non-limiting Examples, with reference to the accompanying drawings in which:

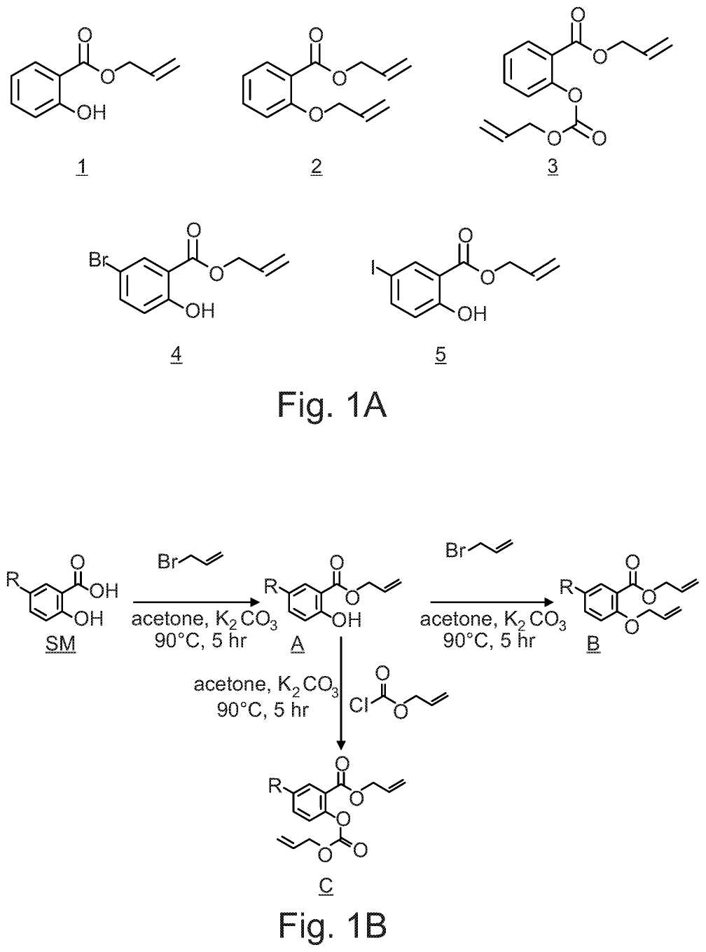

FIG. 1A shows the chemical structures of the salicylic acid derived monomers for use in resin compositions according to Examples of the invention;

FIG. 1B is a synthetic route to the monomers of FIG. 1A;

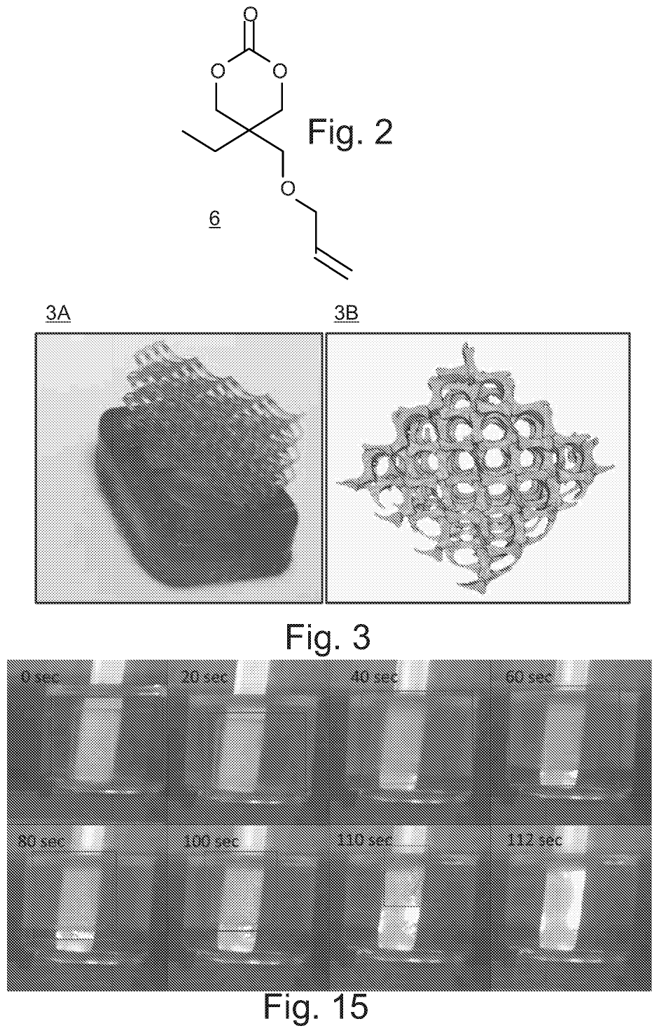

FIG. 2 is the chemical structure of a carbonate monomer used to fabricate oligomers, for use in resin compositions according to Examples of the invention;

FIG. 3 is a 3D printed object and a CT scan of the object fabricated using a resin composition according to an example of the invention;

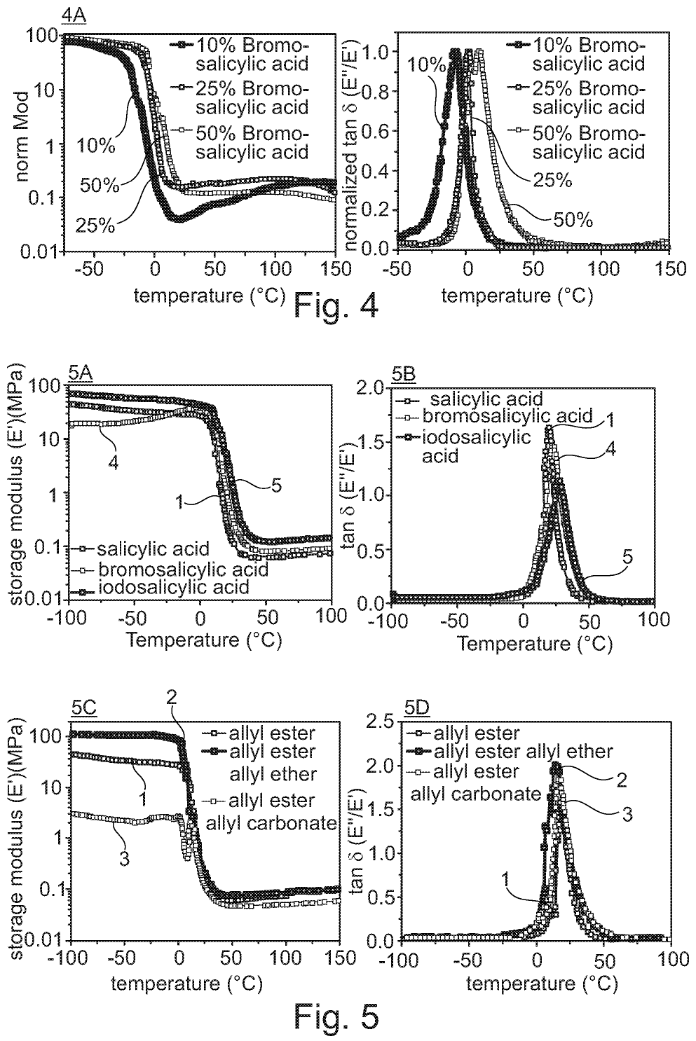

FIG. 4 shows thermomechanical analysis of polymers fabricated using resin compositions with different amounts of salicylic acid derived monomer;

FIG. 5 shows thermomechanical analysis of polymers fabricated using resin compositions with different salicylic acid derived monomers;

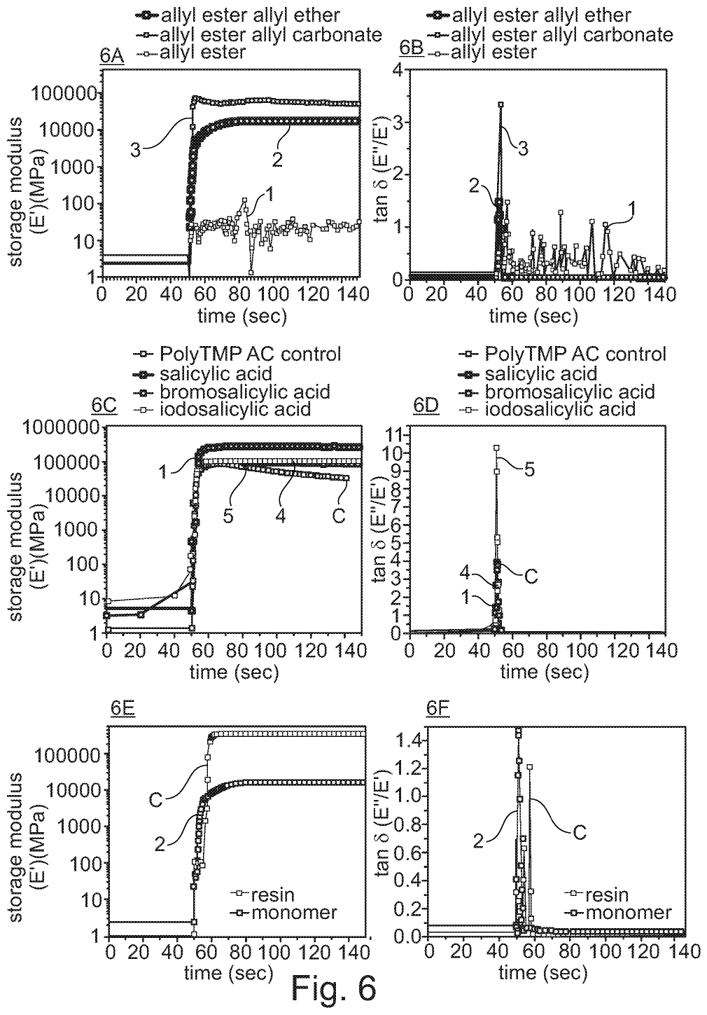

FIG. 6 shows photorheology analysis of resin compositions comprising different salicylic acid derived monomers;

FIG. 7 is photorheology analysis of a resin composition according to the invention;

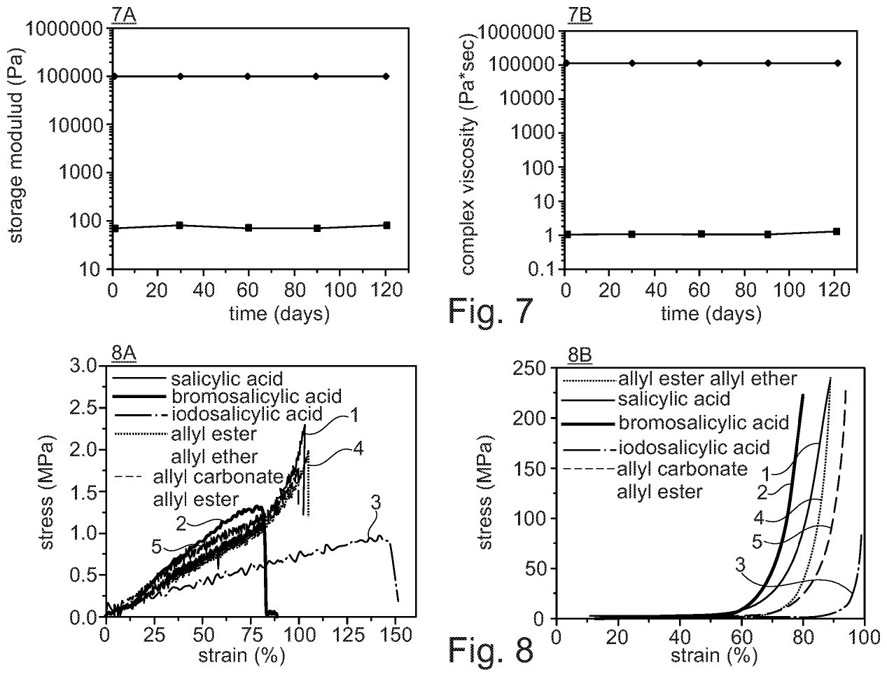

FIG. 8 is uniaxial tensile testing of printed porous scaffolds fabricated using resin compositions containing different salicylic acid derived monomers according to the invention;

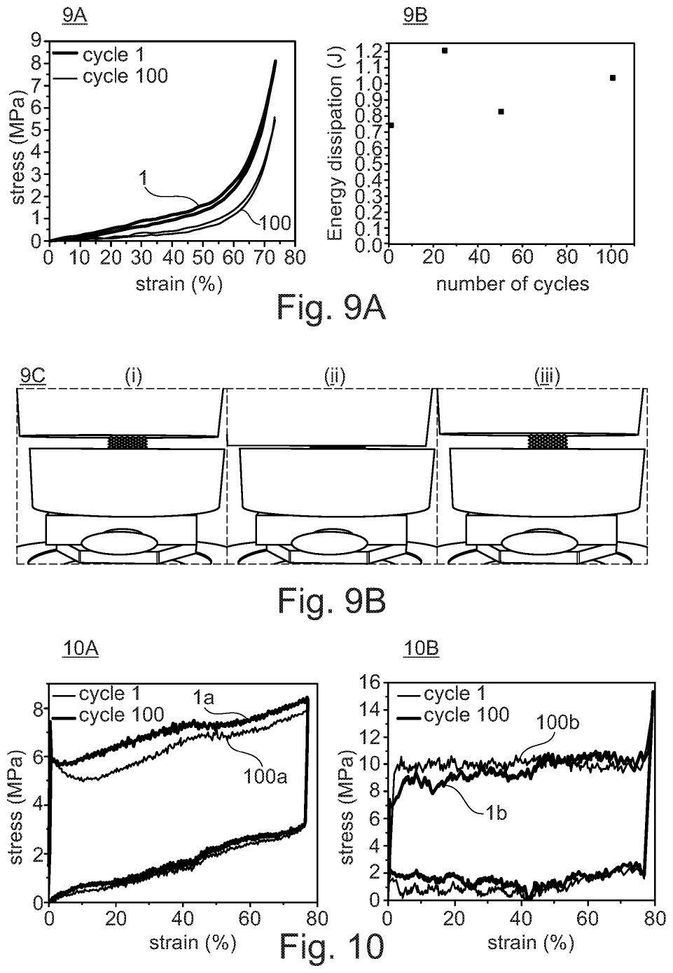

FIG. 9A is a graph showing the cyclic compression of a scaffold fabricated from a crosslinked polymer according to the invention;

FIG. 9B is the apparatus used to carry out the cyclic compression of FIG. 9A;

FIG. 10 is a series of graphs showing the cyclic compression of a scaffold fabricated from a crosslinked polymer according to the invention;

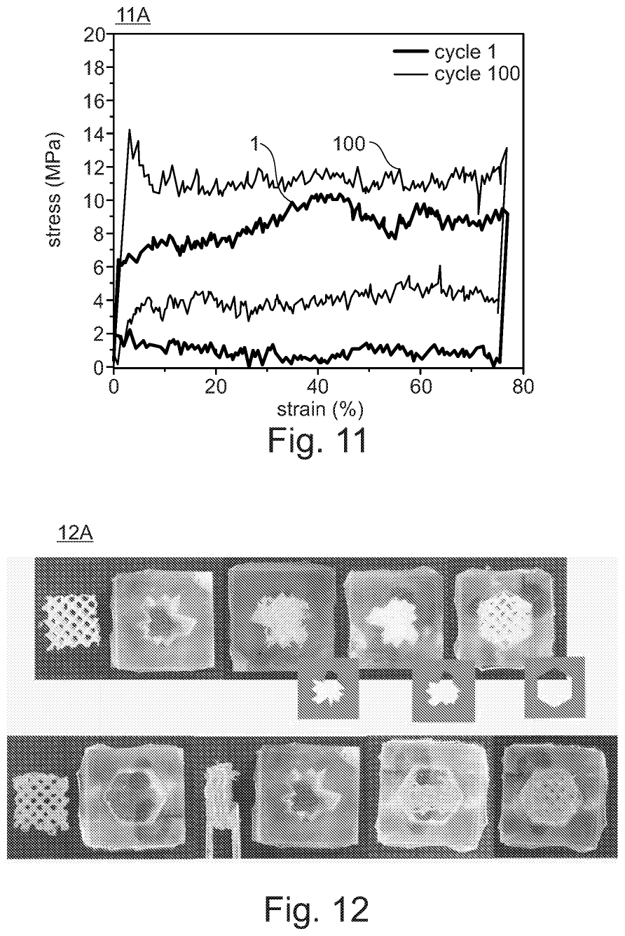

FIG. 11 is a graph showing the cyclic compression of a scaffold fabricated from a crosslinked polymer according to the invention;

FIG. 12 is a series of photographs that demonstrate the shape memory and self-fitting behaviour of scaffolds fabricated from the crosslinked polymer according to the invention;

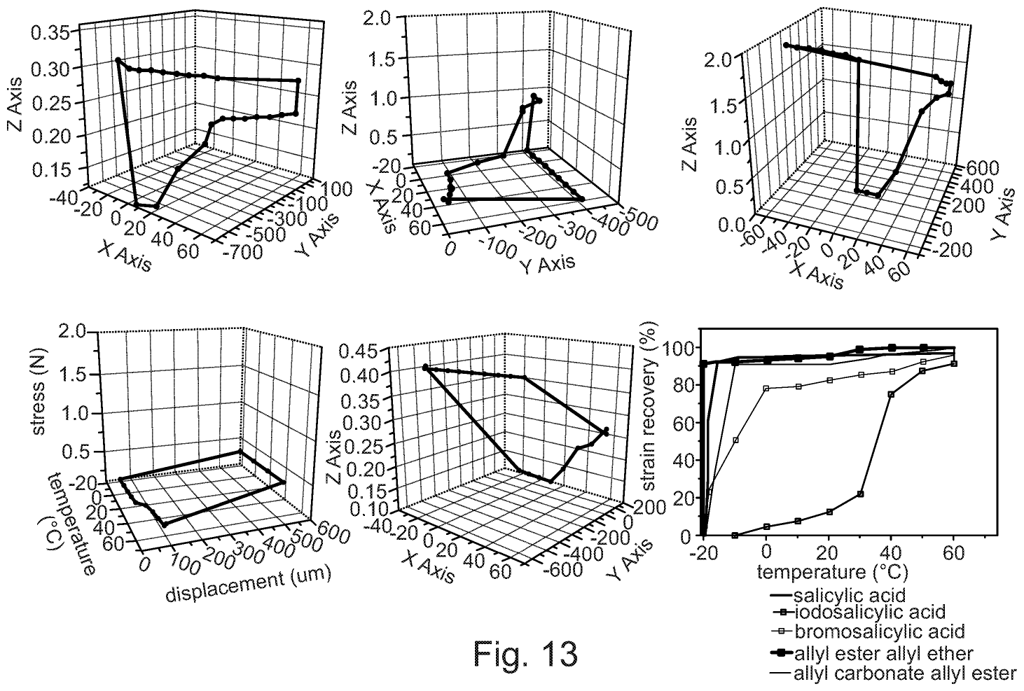

FIG. 13 is a series of graphs illustrating investigation into the strain fixation of crosslinked polymers at different temperatures according to the invention;

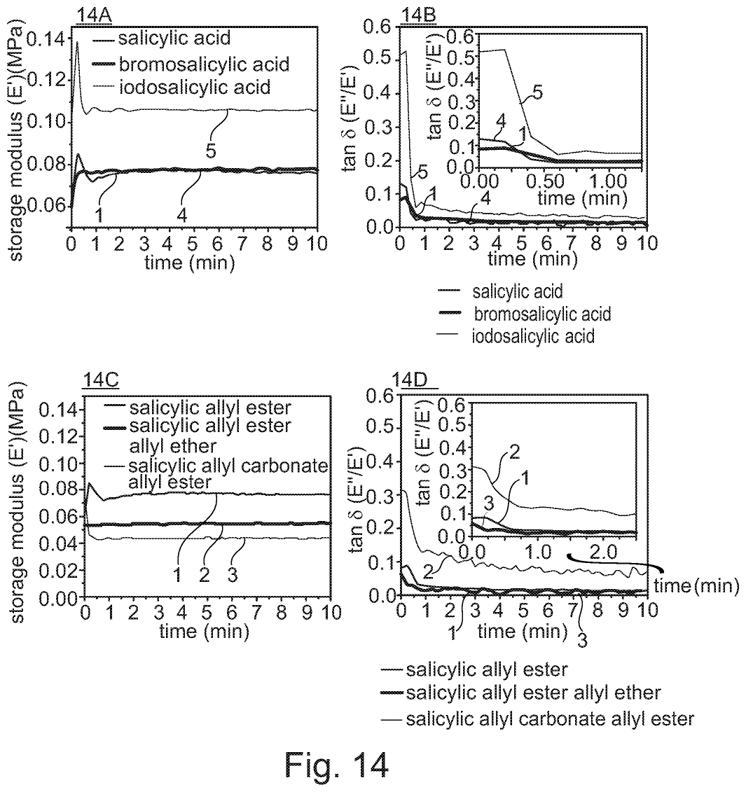

FIG. 14 is a series of graphs illustrating the relaxation kinetics of porous scaffolds fabricated using resin compositions according to the invention;

FIG. 15 is a series of photographs illustrating an example of a surface-eroding polymer film fabricated from a resin composition according to the invention;

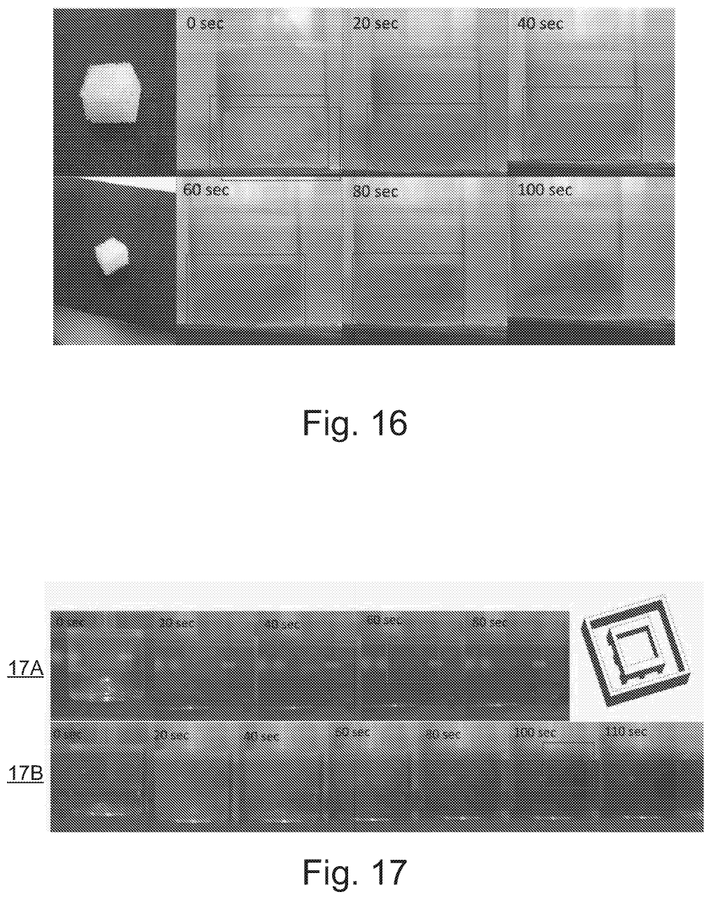

FIG. 16 is a series of photographs illustrating an example of a surface-eroding layered scaffold fabricated from a resin composition according to the invention; and FIG. 17 is a series of photographs illustrating the degradation of scaffolds fabricated using resin compositions according to the invention.

Referring now to FIG. 1A, there is shown salicylic acid derived Monomers 1 to 5 for use in the synthesis of resins and polymers according to the invention.

Referring also to FIG. 1B, there is shown a synthetic route 1 used for the synthesis of Monomers 1 to 5. There is shown the salicylic acid derived starting material SM comprising an R group, wherein the R group represents one of hydrogen, bromine, or iodine. There is also shown Monomer A, which represents Monomer 1 wherein R=H, Monomer 4 wherein R=Br, and Monomer 5 wherein R=I. There is also shown Monomers B and C, which represent Monomer 2 and Monomer 3 respectively wherein R=H.

The synthesis of the Monomers 1 to 5 was achieved via simple reactions of alkyl halides with the alcohol and carboxylic acid sites available on the salicylic acid derived starting material SM. Wherein R=H of SM, a sequential synthesis was used to first form the allyl ester group to produce Monomer 1, which was subsequently used as the starting material to form Monomer 2 and Monomer 3.

Halogenated Monomers 4 and 5 were made in the same manner using a halogenated salicylic acid derived starting material SM wherein R=Br (Monomer 4) or R=I (Monomer 5).

### Procedure for the Synthesis of Monomer 1

Salicylic acid SM (R=H) (100 g, 0.724 mol) was dissolved in acetone (250 mL) in a round bottom flask, to which allyl bromide (122.64 g, 1.014 mol) was added as a single unit. The mixture was heated to reflux and held isothermal for 1 hour prior to the addition of potassium carbonate (70.04 g, 0.507 mol), over the course of 3 hours. The mixture was then allowed to reflux over 12 hours, at which point the remaining acetone was blown off. The solid white crude product was taken up in ethyl acetate and washed with 1 M HCl three times, followed by brine. Monomer 1 was collected as a colourless oil (123.3 g, 0.692 mol) in a 95.6% yield.

### Procedure for the Synthesis of Monomer 2

Monomer 1 (50 g, 0.280 mol) was added to a round bottom flask along with allyl bromide (47.56 g, 0.40 mol) and acetone (100 mL), and was heated to reflux. Potassium carbonate (56.03 g, 0.40 mol) was added over the course of three hours, after which the mixture was allowed to reflux for 12 hours prior to removal of the remaining acetone. The solid crude product was taken up in ethyl acetate, washed with 1 M HCl three times, and once with brine. Monomer 2 was collected by concentration to form a colourless oil (73.1 g, 0.335 mol) in a 92.6% yield.

### Procedure for the Synthesis of Monomer 3

Monomer 1 (50 g, 0.280 mol) was added to a round bottom flask in acetone (100 mL), with allyl chloroformate (47.2 g, 0.391 mol) added dropwise at room temperature. The mixture was heated to reflux, and potassium carbonate (56.03 g, 0.40 mol) was added over the course of five hours after which the mixture was allowed to reflux for another 12 hours. Residual acetone was blown off, and the crude product was dissolved in ethyl acetate, washed three times with 1 M HCl and once with brine. Monomer 3 was collected by concentration to form a colourless oil (69.05 g, 0.263 mol) in a 94.1% yield.

### Procedure for the Synthesis of Monomer 4

Salicylic acid SM (R=Br) (25 g, 0.115 mol) was dissolved in acetone (50 mL) in a round bottom flask, to which allyl bromide (19.51 g, 0.161 mol) was added as a single unit. The mixture was heated to reflux and held isothermal for 1 hour prior to the addition of potassium carbonate (22.29 g, 0.161 mol), over the course of 3 hours. The mixture was then allowed to reflux over 12 hours, at which point the remaining acetone was blown off. The solid white crude product was taken up in ethyl acetate and washed with 1 M HCl three times, followed by brine. Monomer 4 was collected as a colourless oil (25.72 g, 0.090 mol) in a 97.1% yield.

### Procedure for the Synthesis of Monomer 5

Salicylic acid SM (R=I) (25 g, 0.095 mol) was dissolved in acetone (50 mL) in a round bottom flask, to which allyl bromide (16.09 g, 0.133 mol) was added as a single unit. The mixture was heated to reflux and held isothermal for 1 hour prior to the addition of potassium carbonate (16.05 g, 0.133 mol), over the course of 3 hours. The mixture was then allowed to reflux over 12 hours, at which point the remaining acetone was blown off. The solid white crude product was taken up in ethyl acetate and washed with 1 M HCl three times, followed by brine. Monomer 5 was collected as a colourless oil (27.60 g, 0.091 mol) in a 95.6% yield.

Referring now to FIG. 2, there is shown Monomer 6, which was synthesised in one step in the following procedure. Monomer 6 is a cyclic carbonate.

### Procedure for the Synthesis of Monomer 6

Trimethylolpropane allyl ether (112.1 g, 0.64 mol) was dissolved in tetrahydrofuran (THF) (500 mL), followed by the addition of ethyl chloroformate as a single volume (142.7 g, 1.31 mol). The solution was cooled to 0° C. over 1 hour. Triethylamine (140.1 g, 1.38 mol) was then added dropwise over 2 hours. The solid product was filtered after 24 hours and concentrated down. The product was dissolved in ethyl acetate and washed three times with 1M HCl and once with brine to afford Monomer 6.

Monomer 6 was polymerised to form low viscosity oligomers comprising allyl groups in the following procedure.

### Procedure for the Synthesis of Oligomers of Monomer 6

Monomer M6 (20 g, 0.10 mol) was dissolved in dichloromethane (50 mL) under a vacuum that was initially evacuated for 60 seconds. DBU (30 μl) and water (7.5 μl) were added sequentially. The solution was allowed to react for 24 hours. The solution was then concentrated, precipitated in cold hexane, and filtered using ethyl acetate, followed by concentration of the mixture to produce oligomers.

The Oligomers of Monomer 6 were reacted with one or more of Monomers 1, 4, and 5 to form resin compositions comprising oligomers with pendant salicylic acid derived groups, according to Examples of the invention.

### Synthesis of Resin Compositions Using Monomer 1, Monomer 4, or Monomer 5

Monomer 1, Monomer 4, or Monomer 5 (26 g) was added to a beaker along with the oligomers of Monomer 6 (15 g) and a crosslinker (15 g) having the structure shown below as (A). A four-armed thiol, e.g. pentaerythritol tetrakis(3-mercaptopropionate), (46.99 g) was added along with propylene carbonate (21 g) to reduce viscosity further. The resulting resin composition was mixed thoroughly and stored in brown glass when not in use. The resin compositions 1, 2, and 3 (corresponding to Monomers 1, 4, and 5 respectively) each comprised 25 wt. % pendant salicylic acid.

Irgacure 819 (0.25 g, 0.25 wt. %) and paprika extract (0.22 g, 0.22 wt. % inhibitor) were mixed. Acetone (5 mL) was added to aid in dissolution of initiator. The resulting mixture was mixed until dissolved, and then was added directly to a resin composition 1, 2, 3. The acetone was allowed to evaporate over the course of 12 hours before the resin composition was used.

### Crosslinker (A):

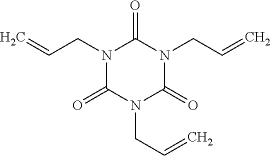 [SMILES: C=CCn1c(=O)n(CC=C)c(=O)n(CC=C)c1=O]

Monomers 2 and 3 were used for form resin compositions with in-chain salicylic acid derived groups according to the invention. It is also possible to use the Oligomers of Monomer 6 to react with one or more of Monomers 2 and 3 to form resin compositions comprising oligomers with in-chain salicylic acid derived groups, according to the invention.

### Synthesis of Resin Compositions Using Monomer 2

Monomer 2 (60 g) was added to a beaker. A four-armed thiol, e.g. pentaerythritol tetrakis(3-mercaptopropionate), (64.2 g) was added, and the solution was mixed until homogenous to form resin composition 4.

Irgacure 819 (photoinitiator, 0.3 g) was added along with paprika extract (photoinhibitor, 0.3% wt). Acetone (5 mL) was added to aid in the dissolution and dispersion of the initiator and inhibitor respectively.

The acetone was removed prior to printing. Propylene carbonate (10 wt. %) was added to further reduce the resin viscosity.

### Synthesis of Resin Compositions Using Monomer 3

Monomer 3 (60.0 g) added to a beaker. A four-armed thiol, e.g. pentaerythritol tetrakis(3-mercaptopropionate), (53.4 g) was added and the solution was mixed until homogenous to form resin composition 5.

Irgacure 819 (photoinitiator, 0.25 g) was added along with paprika extract (photoinhibitor, 0.2% wt). Acetone (5 mL) was added to aid in the dissolution and dispersion of the initiator and inhibitor respectively.

The acetone was removed prior to printing. Propylene carbonate (15 wt. %) was added to further reduce the resin viscosity.

The resin compositions 1 to 5 are photocrosslinkable. These were printed into objects using 3D printing with a UV light.

### Analysis of the Structure and Mechanical Properties of the 3D Printed Objects

Monomer 6 (a cyclic carbonate monomer) was used in the synthesis of oligomers comprising salicylic acid derived monomers for use in making photocrosslinkable resin compositions according to the invention.

Screening studies of the resin compositions according to the invention were undertaken to determine the photoreactivity of the resin compositions and to determine the thermomechanical behaviours of their crosslinked products.

Referring now to FIG. 3, there is shown a 3D printed object 3A and a CT scan of the object 3B fabricated using the photocrosslinkable resin compositions according to the invention.

Referring also to FIG. 4, there is shown a representative thermomechanical analysis 4A and normalised version 4B of resin composition comprising different quantities (10, 25, and 50 mol. %) of Monomer 4 of the total resin composition according to the invention. This was used to determine the resin composition for subsequent 3D printing of salicylic-acid derivative biomaterials scaffolds wherein the number of films (n) examined was n=3.

It was found that polymers comprising 50 mol % of Monomer 4 had the most desirable properties because the highest thermal transitions were observed for this amount. In addition, there are limitations with gelation at concentrations above ˜50 mol %.

Referring also to FIG. 5, there is shown graphs 5A to 5D illustrating thermomechanical analysis of polymers comprising Monomers M1 to M5 according to the invention.

It was found that incorporation of salicylic acid derived Monomers M1 to M5 into the polymer comprising oligomers of Monomer M6 resulted in increased glass transition temperatures with increasing concentration. It was found that storage moduli were only minimally varied with statistically significant differences. It was found that the glass transition temperature of Monomer M4 (wherein R=Br) was lower than for Monomer M5 (wherein R=I) at the same concentration within the oligomer. The glass transition temperature of both Monomer 4 and Monomer 5 was higher than that of the non-halogenated Monomer 1, SM (R=H).

The most dramatic changes were found with polymers fabricated using resin compositions comprising Monomers 2 and 3 with in-chain salicylic acid derivatives, as opposed to the polymers fabricated using resin compositions comprising Monomers 1, 4, and 5 with pendant species.

Polymers fabricated using resin compositions comprising Monomer 1 displayed a lower glassy storage modulus compared with polymers fabricated resin compositions comprising Monomer 2. Both polymers fabricated using resin compositions comprising Monomer 1 and

Monomer 2 were more than an order of magnitude more glassy relative to the polymer fabricated using resin compositions comprising Monomer 3.

Interestingly, the glass transition temperatures were not significantly different between polymers according to the invention as determined by both the tan ∂ and the storage moduli inflection point. All rubbery values were similar in the examined functional groups; polymers comprising Monomer 2 displayed the greatest change (˜3*10³) while Monomer 3 displayed less than 2 orders of magnitude reduction of storage moduli.

Referring now to FIG. 6, there is shown data from photorheology analysis displaying storage moduli and tan ∂ over irradiation time in plots 6A to 6F, which were conducted to examine the crosslinking kinetics of the printing process. Plots 6A and 6B show a comparison of the photorheology of the difference in functional groups between the Monomers 1 to 3. Plots 6C and 6D show a comparison of the photorheology of resin compositions comprising Monomers 1, 4, and 5, and a control (C) comprising no monomer.

Plots 6E and 6F shows a comparison between the photorheology of Monomer 2 in the presence (2) and absence (C) of the oligomer of Monomer 6.

The monomer, e.g. one of Monomer 1 to 5, was mixed with stoichiometric amounts of 4-arm thiol and a photoinitiator for comparison of the rate of gelation as determined by phase transitions within the resin composition. Resin storage modulus and tan ∂ were used as metrics for calculating the rate of gelation.

The functionality of the monomer did not appear to influence the rate of gelation. Monomer 1 (a pendant species) could not form a network and could only 4-arm low molecular weight species. Therefore, resin compositions comprising Monomer 1 did not display a specific phase transition and nor was the storage modulus enhanced by irradiation. This is in contrast to resin compositions comprising Monomer 2 and Monomer 3, in which the phase transition was found to take place within 5 seconds of exposure of light in the printing process.

All resin compositions comprising one of Monomers 1 to 5 optionally along with the oligomers of Monomer 6 displayed rapid and efficient crosslinking without significant variation in gelation time (<5 sec).

As a comparison, the Monomer 2 was incorporated into the resin and was also mixed stoichiometrically with only 4-arm thiol for examining the role of the polycarbonate oligomer of Monomer 6 and diluent in gelation kinetics. As shown in the rheology plots 6E to 6F, Monomer 2 displayed more rapid crosslinking, which is expected due to the concentration (no diluent) as well as more available chain ends in close proximity of the thiol end groups. However, Monomer 2 was not as rapidly curing with regards to the final storage modulus value (order of magnitude difference in value), indicating a need for the oligomer to stabilize larger 3D objects during photoprinting.

Referring now to FIG. 7, there is shown analysis of the photorhelology (7A) and the storage life (7B) of a resin composition according to the invention.

Photorheology experiments were used to provide a straight-line approximation of the initial (pre-photoirradiation) and final (after gelation occurred) storage modulus and complex viscosity of a resin composition of the invention over time.

It is shown that the resin compositions of the invention display pot-lifes in excess of 120 days when stored in brown glass containers. Even with the photoinitiators present, these resin compositions of the invention appear to be very stable and promising for continuous process of biomaterial scaffolds.

Referring now to FIG. 8, there is shown uniaxial tensile testing of printed porous scaffold dogbones fabricated using polymers comprising Monomers 1 to 5 at ambient conditions tested at 5 mm/min at 37° C. (8A), and compression testing to failure at ambient conditions, compressed at 3 mm/min (8B).

Polymers comprising Monomers 1 to 5 were printed using resin compositions according to the invention. These printed porous scaffolds are intended as implantable materials for soft-tissue applications, meaning that while structural support is not crucial, an ideal scaffold will display approximately 1 month of elasticity prior to degradation. Specifically, scaffolds should be able to return energy applied in the form of deformation over this time period, as this would similar to how native tissue would respond.

Initial mechanical analysis was performed using tensile testing of printed dogbones (modified ASTM Type IV) and compression of porous scaffolds. Pendant salicylic acid derivatives (polymers comprising Monomers 1, 4, or 5) resulted in elastomeric materials possessing strains to failure of approximately 100% regardless of halogen species (R=H, Br, I). Polymers comprising monomer 2 was found to be the most brittle tensile and compressive sample, with strain to failure occurring at approximately 80% in tension and 140% compression. However, in compression an inflection point is found at ˜70%, indicating the onset of failure in this region when the sample is dry.

Referring now to FIG. 9A, there is shown a graph 9A showing the cyclic compression of a scaffold fabricated from a crosslinked polymer according to the invention comprising Monomer 5 (wherein R=I). The cyclic compression was recorded at ambient conditions over 100 cycles. Cycle 1 and Cycle 100 are labelled. In FIG. 9A, there is also shown graph 9B, which illustrates the corresponding absorbed energy at each cycle (B) wherein the number of samples (n) is (n=3).

Referring also to FIG. 9B, there is shown the apparatus 9C used to carry out the cyclic compression of the scaffold S. The apparatus 9C is shown in an initial non-compressed state (i), a compressed state (ii), and a second non-compressed state (iii). This was repeated for 100 cycles to test the compressive stress of the scaffold.

The initial cyclic examinations were performed at room temperature and ambient conditions, where the scaffolds were compressed to approximately 80%, determined as the failure region from single compression and tensile tests to failure. Representative compression behaviour is presented using a crosslinked polymer according to the invention comprising Monomer 5 (wherein R=I), where the peak compressive stress was found to decrease over the course of 100 cycles although the energy absorbed was found to remain relatively stable. The same behaviour has been demonstrated with Monomers 1 to 3.

Referring now to FIG. 10, there is shown graph 10A and 10B showing the cyclic compression of a scaffold fabricated from a crosslinked polymer. The graph 10A shows the cyclic compression of a scaffold fabricated from a crosslinked polymer comprising only oligomer. The graph 10B shows the cyclic compression of a scaffold according to the invention comprising Monomer 2. The cyclic compression was recorded at 37° C. in PBS (phosphate-buffered saline) over 100 cycles. Cycle 1a, 1b and Cycle 100a, 100b are labelled respectively.

It is shown that, importantly, upon immersion of the scaffold over the course of 1 month, the scaffold displayed robust mechanical behaviours. This was observed for all other crosslinked polymers according to the invention that were fabricated into scaffolds.

It was shown that the loss in mechanical behaviour displayed at ambient conditions (FIG. 9A) was not found to be as significant at 1 day (FIG. 10A), and at 1 month there were no differences (FIG. 10B). Interestingly, the maximum compressive stress was found to have increased, although the rapid elastic response of the materials was decreased at this time, as determined by the change in the recovery shape. This may be due to the increased weight of the material as a result of water influx.

In these tests, cycle 1 displayed reduced stress prior to deformation compared to cycle 100. Interestingly, these materials did not fail in this region, nor was there strut rearrangement or macroscopic changes to the material (failures). Without wishing to be bound by any particularly theory, it is thought that the enhanced mechanical response may be due to additional PBS infiltration, producing a more gel-like material which does not toughen with increased strain. This behaviour, coupled with the stereotypical J-shaped stress-strain relationship to failure, indicates how promising these materials are for biomaterials.

Referring now to FIG. 11, there is shown a graph 11A showing the cyclic compression of a scaffold fabricated from a crosslinked polymer according to the invention comprising Monomer 3. The cyclic compression was recorded at 37° C. in PBS (phosphate-buffered saline) over 100 cycles. Cycle 1 and Cycle 100 are labelled. This shows the initial and degradation-dependent mechanical profiles for cyclic testing of a crosslinked polymer comprising Monomer 2, with data taken at the 100^{th} compressive cycle.

This behaviour (shown in FIGS. 10 and 11) is representative of that for crosslinked polymers comprising Monomers 1 to 5 according to the invention.

The crosslinked polymer according to the invention comprising Monomer 3 were the most elastomeric materials produced. This material possibly demonstrates the greatest promise for soft tissue engineering applications. Cyclic compressive testing (not shown) indicated that the crosslinked polymer comprising Monomer 3 is stable over a 1 month period, and that its behaviour is consistent up to 50° C.

Referring now to FIG. 12, there is shown a series of photographs that demonstrate the shape memory and self-fitting behaviour of scaffolds fabricated from the crosslinked polymer according to the invention. This behaviour is representative of that for crosslinked polymers comprising Monomers 1 to 5 according to the invention. The same porous scaffolds were used to measure the mechanical compression of the materials, as shown in FIGS. 10 and 11.

Referring also to FIG. 13, there is shown a series of graphs illustrating investigation into the strain fixation of crosslinked polymers according to the invention at −20° C.

It was found that only −20° C. displaying any strain fixation. The shape memory effect was interesting to examine, as monomers according to the invention could display some dipole-dipole interactions at the carbonyls, possibly providing some temporary crosslinking sites for secondary shape fixation. Surprisingly, the resin compositions comprising Monomer 1 (comprising a pendant salicylic acid moiety) did not display any shape fixation, even when the glass transition temperature T_{g} (measured by tan ∂) was more than 20° C. above the fixation temperature of −20° C. Crosslinked polymers comprising Monomer 4 (R=Br) or Monomer 5 (R=I) did display some strain fixation, with polymers comprising Monomer 5 possessing strain fixation up to 30° C.

Interestingly, the various functional groups of Monomers 1 to 5 did not impact the strain fixation of the crosslinked polymer significantly, despite all possessing onset temperatures (full width half max of the tan ∂ peak) more than 20° C. above −20° C. As a note, polymers comprising Monomer 5 displayed an onset temperature of approximately 10° C. compared with 0° C. for Monomers 1 to 3. This shows that these materials are more likely to self-fit to irregularly shape voids without requiring additional stimuli or external assistance.

Referring also to FIG. 14, there is shown graphs 14A to 14D illustrating the relaxation kinetics of porous scaffolds fabricated using resin compositions according to the invention. The data was recorded when the scaffolds were immersed in PBS at 37° C. This demonstrates the effect of the composition of the monomer on the rate and extent of final polymer chain relaxation. The data is labelled according to monomer used in the resin composition used to fabricate the scaffold, i.e. Monomer 1, 2, 3, 4, or 5.

The behaviour described above is also supported by the extent of relaxation the scaffolds display prior to and immediately after immersion in PBS, where only polymers comprising Monomer 5 display significant relaxation.

### Degradation and Surface Erosion Studies

Degradation and drug release studies have demonstrated that the resin compositions according to the invention used to produce surface eroding parts. This has been demonstrated with films as well as printed scaffolds.

Referring now to FIG. 15, there is shown a series of photographs illustrating an example of a surface-eroding polymer film fabricated from a resin composition according to the invention. The surface erosion was measured over 0 to 112 seconds in 5M NaOH at 37° C.

Referring now to FIG. 16, there is shown a series of photographs illustrating an example of a surface-eroding layered scaffold fabricated from a resin composition according to the invention. The scaffold was immersed in 5M NaOH at 37° C. and the surface erosion was measured over 0 to 100 seconds. This demonstrates surface erosion in 3D.

Advantageously, different parts of the scaffold may be fabricated from resin compositions comprising different monomers, e.g. Monomer 1, 2, 3, 4, and/or 5. This enables selective degradation of specific sections of the scaffold to occur such that different profiles of mass loss and NSAID release are observed.

Referring now to FIG. 17, there is shown a series of photographs illustrating the degradation of scaffolds fabricated using resin compositions according to the invention. The multi-layered scaffolds were fabricated using resin compositions according to the invention and further comprising one of fluorescein, Nile blue, or salicylic acid.

Series 17A was fabricated using a resin composition comprising a pendant salicylic acid monomer. Series 17B was using a resin composition comprising an in-chain salicylic acid monomer. Each scaffold had the following structure, described from the outermost layer to the innermost layer: (i) salicylic acid; (ii) fluorescein; (iii) salicylic acid; (iv) Nile blue core. The studies were conducted in 5M NaOH at 37° C.

It was found that the pendant salicylic acid-derived matrix (17A) displayed rapid release while the in-chain salicylic acid-based monomers (17B) display more sustained, slower release. This demonstrates the ability to incorporated small molecule drugs into the polymer part during printing, where the release rate of the part may be controlled by tuning the composition, e.g. in a multi-layered structure.

It will be appreciated by those skilled in the art that several variations to the aforementioned embodiments are envisaged without departing from the scope of the invention. For example, the monomers need not comprise alkene functionality. Any functionality described in WO2018229456 (e.g. epoxides, alkynes, azides) may be used in the present invention.

It will also be appreciated by those skilled in the art that any number of combinations of the aforementioned features and/or those shown in the appended drawings provide clear advantages over the prior art and are therefore within the scope of the invention described herein.

1-25. (canceled)

26. A photocurable resin composition, the photocurable resin composition comprising a photoinitiator, optionally an oligomer comprising at least one alkene moiety, and a drug-derived monomer having at least one photo-crosslinkable moiety.

27. A photocurable resin composition according to claim 26, wherein the drug-derived monomer comprises at least one group comprising an alkene moiety and wherein the drug-derived monomer is a monomer derived from one or more of the following NSAID classes: salicylates, propionic acid derivatives, acetic acid derivatives, enolic acid (oxicam) derivatives, anthranilic acid derivatives (fenamates), selective COX-2 inhibitors, sulfonanilides, and others.

28. A photocurable resin composition, the photocurable resin composition comprising a photoinitiator, optionally an oligomer comprising at least one alkene moiety, and an NSAID-derived monomer, the NSAID-derived monomer having the following general formula: 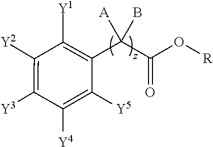
wherein Y¹, Y², Y³, Y⁴ and Y⁵ independently represent hydrogen, a halogen, or comprises an aliphatic group or chain which may be straight, branched or cyclic, an aromatic group or chain, oxygen, a hydroxyl moiety, or a substituted oxygen moiety such as an ether, ester, ketone or aldehyde, an amide, sulphur, an SH thiol moiety, or a substituted thiol moiety, nitrogen, an NH₂ moiety, or a di- or tri-substituted nitrogen moiety, or Rₓ, C—Rₓ, O—Rₓ, C(O)—Rₓ where Rₓ represents a straight, branched or cyclic alkane, which may comprise one or more hetero atoms or an aryl group;
A and B independently represent a hydrogen atom, a halogen atom, or an alkyl group;
Z is one of 0, 1, 2, 3, 4, 5; and
R comprises an alkene moiety.

29. A photocurable resin composition, the photocurable resin composition comprising a photoinitiator, optionally an oligomer comprising a least one alkene moiety, and a salicylic acid derived monomer, the salicylic acid derived monomer having the following general formula: 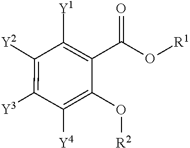
wherein Y¹, Y², Y³, and Y⁴ independently represent hydrogen, an aliphatic group or chain, or an aromatic group or chain, a halogen, a hydroxyl moiety, or a substituted oxygen moiety such as an ether, an SH thiol moiety, or a substituted thiol moiety, an NH₂ moiety, or a di- or tri-substituted nitrogen moiety;
and one or both of R¹ or R² comprise an alkene moiety.

30. A photocurable resin composition according to claim 29, wherein the salicylic acid derived monomer has the following general formula: 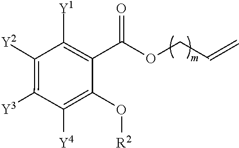
wherein m is a number between 1 and 5.

31. A photocurable resin composition according to claim 30, wherein R² represents hydrogen.

32. A photocurable resin composition according to claim 26, wherein the drug derived monomer has the following general formula: 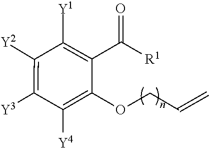
wherein Y¹, Y², Y³, Y⁴ and Y⁵ independently represent hydrogen, a halogen, an aliphatic group or chain which may be straight, branched or cyclic, an aromatic group or chain, oxygen, a hydroxyl moiety, or a substituted oxygen moiety such as an ether, ester, ketone or aldehyde, an amide, sulphur, an SH thiol moiety, or a substituted thiol moiety, nitrogen, an NH₂ moiety, or a di- or tri-substituted nitrogen moiety, or Rₓ, C—Rₓ, O—Rₓ, C(O)—Rₓ where Rₓ represents a straight, branched or cyclic alkane, which may comprise one or more hetero atoms or an aryl group;
and R¹ comprises an alkene moiety;
wherein n is a number between 1 and 5.

33. A photocurable resin composition according to claim 26, wherein the drug derived monomer has the following general formula: 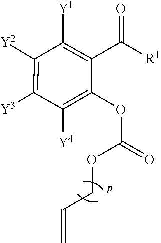
wherein Y¹, Y², Y³, Y⁴ and Y⁵ independently represent hydrogen, a halogen, an aliphatic group or chain which may be straight, branched or cyclic, an aromatic group or chain, oxygen, a hydroxyl moiety, or a substituted oxygen moiety such as an ether, ester, ketone or aldehyde, an amide, sulphur, an SH thiol moiety, or a substituted thiol moiety, nitrogen, an NH₂ moiety, or a di- or tri-substituted nitrogen moiety, or Rₓ, C—Rₓ, O—Rₓ, C(O)—Rₓ where Rₓ represents a straight, branched or cyclic alkane, which may comprise one or more hetero atoms or an aryl group;
and R¹ comprises an alkene moiety;
wherein p is a number between 1 and 5.

34. A photocurable resin composition according to claim 29, wherein Y¹, Y³, and Y⁴ each independently represent hydrogen, and Y² represents one of hydrogen, bromine, or iodine.

35. A photocurable resin composition according to claim 26, comprising more than one distinct NSAID-derived monomer.

36. A photocurable resin composition according to claim 29, wherein the salicylic acid derived monomer is selected from one or more of the following compounds (i) to (v):

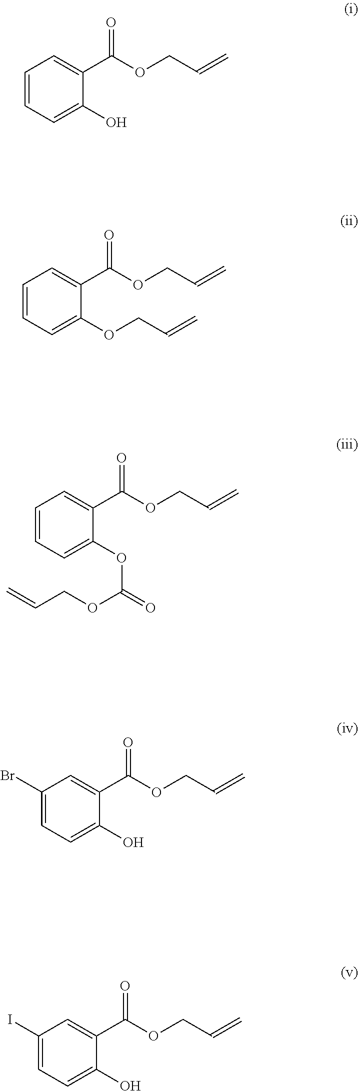

37. A photocurable resin composition according to claim 26, wherein the oligomer comprises one or more polycarbonate linkages.

38. A photocurable resin composition according to claim 37, wherein the oligomer comprises a monomer with the following structure:

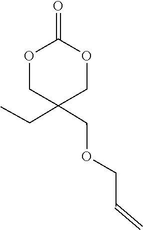

39. A photocurable resin composition according to claim 37, wherein the oligomer comprises between 3 and 100 repeating monomers.

40. A photocurable resin composition according to claim 26, further comprising either (a) a photoinhibitor, (b) a crosslinker, (c) a diluent, or combinations of two or more of (a), (b) and (c).

41. A photocurable resin composition according to claim 26, wherein the sum of alkene moieties on the optional oligomer and photo-crosslinkable moieties on the drug-derived monomer is two or greater.

42. A method of crosslinking a resin composition comprising salicylic acid derived monomers, to form a polymer, the method comprising:
i. providing a photocurable resin composition according to claim 26;
ii. irradiating the resin composition with light.

43. A method according to claim 42, wherein step ii. comprises irradiating the resin composition with UV light and wherein the method is performed by additive manufacturing, such as on a 3D printer.

44. A crosslinked polymer fabricated from the photocurable resin composition of claim 26.

45. A scaffold fabricated according to the crosslinked polymer of claim 44.
